(19) United States
(12) Patent Application Publication
(10) Pub. No.: US 2006/0069441 A1
(43) Pub. Date: Mar. 30, 2006

(54) Posterior approach implant method for assembly of multi-piece artificial spinal disk replacement device in situ

(76) Inventors: James F. Zucherman, San Francisco, CA (US); Ken Y. Hsu, San Francisco, CA (US)

Correspondence Address: FLIESLER MEYER, LLP, FOUR EMBARCADERO CENTER, SUITE 400, SAN FRANCISCO, CA, 94111, US

(21) Appl. No.: 10/979,841

(22) Filed: Nov. 2, 2004

Related U.S. Application Data
(60) Provisional application No. 60/614,181, filed on Sep. 29, 2004.
(60) Provisional application No. 60/614,246, filed on Sep. 29, 2004.
(60) Provisional application No. 60/614,061, filed on Sep. 29, 2004.

(51) Int. Cl. A61F 2/44

(52) U.S. Cl. 623/17.15

(57) ABSTRACT

A posterior approach for intervertebral disk replacement is provided. This technique is particularly suited for assembling a multi-piece artificial spinal disk replacement device in situ in order to alleviate discomfort associated with the spinal column.

### PRIORITY CLAIM

This application claims priority to the following three provisional applications, which are each hereby incorporated by reference in their entirety:

POSTERIOR APPROACH IMPLANT METHOD FOR ASSEMBLY OF A MULTI-PIECE ARTIFICIAL SPINAL DISK REPLACEMENT DEVICE IN SITU, U.S. Provisional Patent Application No. 60/614,181, filed on Sep. 29, 2004, Inventors: James Zucherman and Ken Y. Hsu (Attorney's Docket No. KLYCD-05001 US1);

MULTI-PIECE ARTIFICIAL SPINAL DISK REPLACEMENT DEVICE WITH SELECTABLY POSITIONING ARTICULATING ELEMENT, U.S. Provisional Patent Application No. 60/614,246, filed on Sep. 29, 2004, Inventors: James Zucherman and Ken Y. Hsu (Attorney's Docket No. KLYCD-05001 US2);

MULTI-PIECE ARTIFICIAL SPINAL DISK REPLACEMENT DEVICE WITH MULTI-SEGMENTED SUPPORT PLATES, U.S. Provisional Patent Application No. 60/614,061, filed on Sep. 29, 2004, Inventors: James Zucherman and Ken Y. Hsu (Attorney's Docket No. KLYCD-05001 US3).

### CROSS REFERENCES To RELATED APPLICATIONS

This application is related to the following co-pending applications which are hereby incorporated by reference in their entirety:

MULTI-PIECE ARTIFICIAL SPINAL DISK REPLACEMENT DEVICE WITH SELECTABLY POSITIONING ARTICULATING ELEMENT, U.S. patent application Ser. No. ______, filed on Nov. 2, 2004, Inventors: James Zucherman and Ken Y. Hsu (Attorney's Docket No. KLYCD-05001 US7).

MULTI-PIECE ARTIFICIAL SPINAL DISK REPLACEMENT DEVICE WITH MULTI-SEGMENTED SUPPORT PLATES, U.S. patent application Ser. No. ______, filed on Nov. 2, 2004, Inventors: James Zucherman and Ken Y. Hsu (Attorney's Docket No. KLYCD-05001US8).

### FIELD OF THE INVENTION

This invention relates to multi-piece artificial vertebral disks and techniques for assembling the disks in situ via a posterior approach.

### BACKGROUND OF THE INVENTION

The spinal column is a biomechanical structure composed primarily of ligaments, muscles, vertebrae and intervertebral disks. The biomechanical functions of the spine include: (1) support of the body, which involves the transfer of the weight and the bending movements of the head, trunk and arms to the pelvis and legs, (2) complex physiological motion between these parts, and (3) protection of the spinal cord and nerve roots.

As the present society ages, it is anticipated that there will be an increase in adverse spinal conditions which are characteristic of older people. Pain associated with such conditions can be relieved by medication and/or surgery. Of course, it is desirable to eliminate the need for major surgery for all individuals and in particular for the elderly.

More particularly, over the years, a variety of intervertebral implants have been developed in an effort to relieve the pain associated with degenerative and dysfunctional disk conditions. For example, U.S. Pat. No. 4,349,921 to Kuntz discloses an intervertebral disk prosthesis that consists of two prosthesis parts that are positioned side-by-side between adjacent vertebrae. The two parts together are said to replace the function of a natural intervertebral disk. This patent also discloses that the two parts can be implanted by a posterior approach.

U.S. Pat. No. 4,714,469 to Kenna discloses a spinal implant that fuses vertebrae to the implant. The implant has a rigid body that fits between the vertebrae with a protuberance extending from a vertebral contacting surface and extends into the vertebral body.

U.S. Pat. Nos. 4,772,287 and 4,904,260 both to Ray et al. disclose implanting two prosthetic disc capsules side-by-side into the nucleus of the annulus of a damaged disk. The capsules are filled with a fluid.

U.S. Pat. No. 5,562,736 to Ray et al. discloses a surgical procedure for implanting a prosthetic spinal disk nucleus into a spinal disk space through a posterior side of the annulus.

U.S. Pat. No. 5,258,031 to Salib et al. discloses another prosthetic disk with a ball that fits into a socket.

U.S. Pat. Nos. 5,425,773 and 5,562,738 both to Boyd et al. disclose a disk arthroplasty device for replacement of the spinal disk. A ball-and-socket are provided to enable rotation.

U.S. Pat. No. 5,534,029 to Shima discloses an articulated vertebral body spacer with a pair of upper and lower joint pieces inserted between the vertebrae. An intermediate layer is provided to allow for movement between the upper joint piece and the lower joint piece.

U.S. Pat. No. 5,782,832 to Larsen et al. discloses a two-piece ball-and-socket spinal implant with upper and lower plates for insertion within the intervertebral space.

U.S. Pat. No. 6,156,067 to Bryan et al. discloses a prosthesis having two plates with a nucleus there between.

None of these solutions provides an implant that restores a wide range of natural movement. Moreover, the posterior approach surgical procedures disclosed are limited to implanting relative small devices.

Accordingly, the art is in search of implants for alleviating adverse spinal conditions and for restoring natural movement to the spinal column. In addition, the art is in need of surgical techniques for implanting large devices and especially multiple-piece devices between vertebrae by a posterior approach.

### BRIEF DESCRIPTION OF THE DRAWINGS

FIG. 1A is a posterior elevational partial view of the spinal column.

FIG. 1B is a transaxial view of the spine.

FIG. 2A is a posterior elevational partial view of the spinal column showing the unilateral facet removal.

FIG. 2B shows a transaxial view of the spine after the unilateral facet removal.

FIG. 2C is a posterior elevational partial view of the spinal column showing the removal of a portion of the annulus.

FIG. 3A is the posterior elevational partial view of the spinal column showing the initial insertion of an implant through a posterior annulotomy.

FIG. 3B is the transaxial view of the spine showing the initial insertion of the implant.

FIG. 3C is the posterior elevational partial view of the spinal column showing the positioning of the implant against the end plate or lower surface of the upper vertebra.

FIG. 3D is the transaxial view of the spine showing the positioning of the implant against the upper vertebra.

FIG. 3E is the posterior elevational partial view of the spinal column showing the initial insertion of a second implant through the posterior annulotomy.

FIG. 3F is the posterior elevational partial view of the spinal column showing the insertion of a third implant through the posterior annulotomy wherein the third implant is positioned between the first and second implants.

FIGS. 4, 5, and 6 are the posterior elevational partial views of the spinal column showing the initial insertions of three different sized implants through a posterior annulotomy.

FIG. 7A is a posterior elevational partial view of an assembled multi-piece implant in its neutral position having a first or upper plate, a second or lower plate, and an articular surface between the first and second plates.

FIG. 7B is the plan view of the lower surface of the first plate of the implant.

FIG. 7C is the plan view of the upper surface of the second plate of the implant.

FIG. 7D is the side view of the articular surface.

FIG. 7E is the plan view of the lower surface of the second plate of the implant with the articular surface attached.

FIG. 8 is a block diagram showing the method steps of the posterior implantation of an embodiment of the disclosed implant.

### DETAILED DESCRIPTION OF THE PREFERRED EMBODIMENTS OF THE INVENTION

Embodiments of an apparatus and methods for a posterior approach for intervertebral disk replacement of the present invention are provided. The apparatus and methods are particularly suited for assembling a multi-piece artificial spinal disk replacement device in situ in order to alleviate discomfort associated with the spinal column.

The following description is presented to enable any person skilled in the art to make and use the invention. Various modifications to the embodiments described will be readily apparent to those skilled in the art, and the principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all patents and patent applications cited in this application are incorporated herein by reference.

Other aspects, objects, features and elements of embodiments of the invention are described or evident from the accompanying claims and figures.

In one embodiment, the invention provides a technique for implanting a “large” artificial spinal replacement device or implant via a posterior approach to the spine. The term “large” is meant that the width of the device (or individual pieces that form the device) implanted is longer than both the width and height of a substantially rectangular-shaped opening that is created through the annulus by a posterior annulotomy and through which the device (or individual pieces thereof) is positioned into the nucleus pulposis (or the intervertebral space created by its removal).

The inventive procedure is particularly suited for assembling in situ a multi-piece artificial spinal disk replacement device wherein at least one of the pieces of the device preferably has a width that is longer than both the width and height of the substantially rectangular-shaped opening in the annulus. Accordingly, the individual pieces of the devices are inserted through this opening and the pieces are assembled within the nucleus pulposis (or the intervertebral space created by its removal) to form the multi-piece device. By “multi-piece” device is meant a spinal disk replacement device having at least two parts or pieces that cooperate with each other in distributing weight through the spine and simulating motion of the spine. Preferred multi-piece devices when assembled have the pieces that are positioned one on top of the other along a vertical axis.

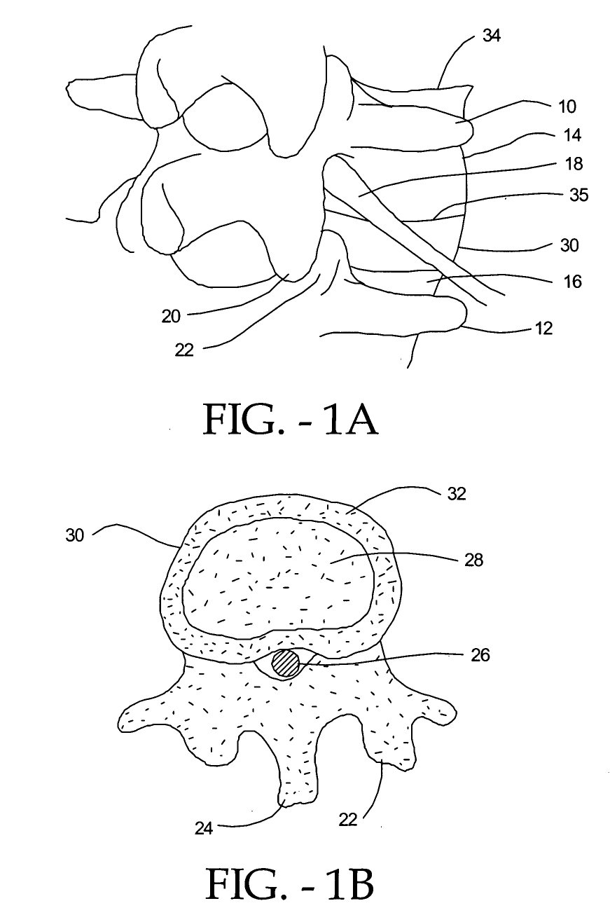

Referring to FIGS. 1A and 1B, the spinal column includes successive vertebrae 10 and 12 with vertebral bodies 14 and 16, respectively. A vertebral disk 30, which is situated between the vertebral bodies 14, 16, includes an outer annulus fibrosis 32 and an inner nucleus pulposis 28. The annulus fibrosis 32 is a ligamentous ring which binds the adjacent vertebrae 10, 12 together. The body 14 of the vertebra 10 has concave upper and lower surfaces 34, 35, respectively, with raised marginal edges. A layer of cartilage covers the body surfaces 34, 35. The neural canal contains the cauda equina or spinal cord 26. Various processes 24 extend from the body and these shield the spinal cord 26 and provide attachment sites for muscles. Nerves 18 extend from the spinal cord 26 in the interstices of the processes. The annulus fibrosis 32 along with the facet joints 20, 22 restrict the torsional motion or twisting between vertebrae.

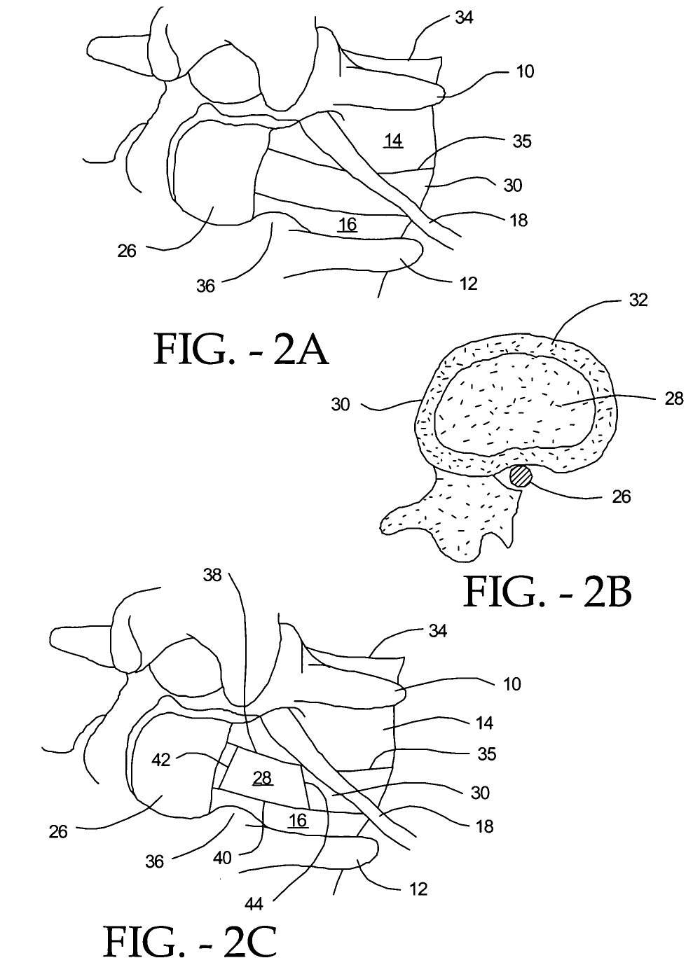

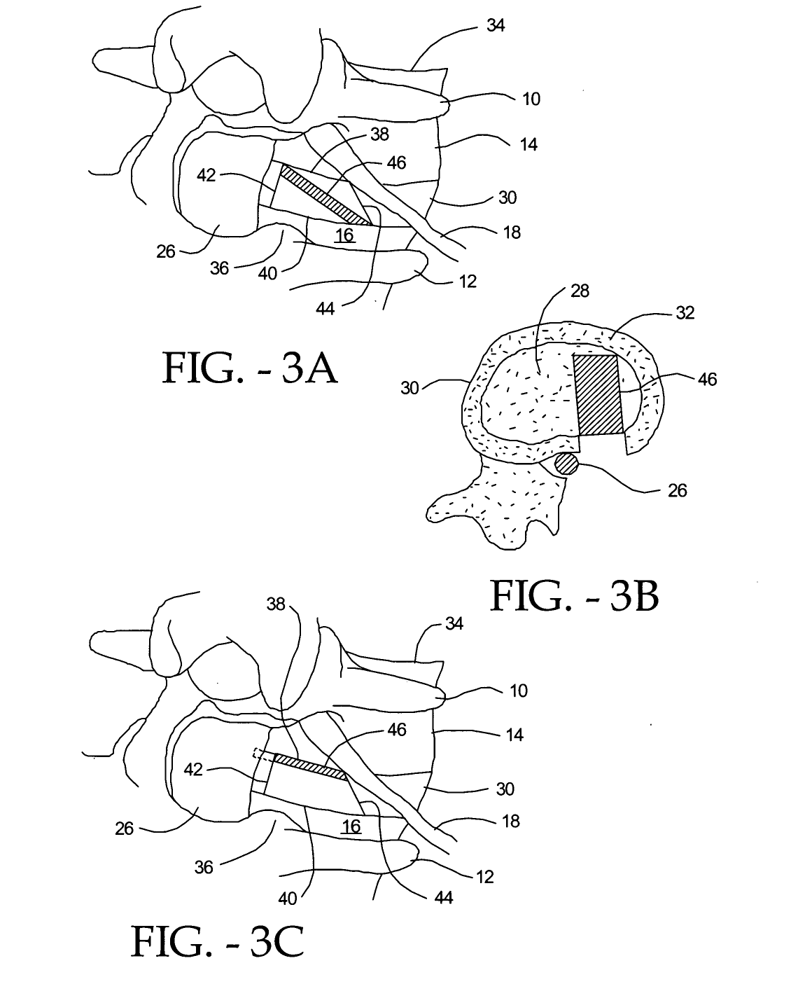

The steps for replacing the nucleus pulposis of the disk through a posterior approach with an artificial spinal disk replacement device are shown in FIGS. 2A through 2C, and FIGS. 3A through 3F. FIGS. 2A and 2B show the exposed affected region of the spine posteriorly after unilateral facet removal from vertebrae 10 and 12. Pedicle 36 of vertebra 12 may be left in intact.

Following the unilateral facet removal, as shown in FIG. 2C, the surgeon performs an annulotomy whereby a flap (not shown) is cut from the posterior annulus 32 to expose the nucleus pulposis 28. As is apparent, the opening is substantially rectangular with upper and lower sides 38 and 40, and lateral sides 42 and 44. The upper side 38 is preferably substantially flushed with the lower surface of vertebral body 14 and the lower side 40 is preferably substantially flushed with the upper surface of vertebral body 16. The upper and lower surfaces of the vertebral bodies are also referred to as end plates. During the procedure, the caudal equina 26 can be moved by the surgeon to one side by a nerve root retractor. As shown, the inner side 42 of the opening is preferably near the midsiggital plane of the disk 30. Next, a portion of the nucleus pulposus corresponding to the space that will be occupied by the assembled multi-piece is removed.

In the case where the device to be implanted does not include any piece (or pieces) that has a particularly long width vis-à-vis the dimensions of the disk being treated or replaced, the dimensions of the opening created by the annulotomy can be such that the diagonal of the opening will accommodate the a device as shown in FIG. 3A. The first piece 46 of the multi-piece device is inserted through the opening of the posterior annulus with the with of the first piece being positioned along the diagonal of the opening. The first piece 46 is inserted into the disk in the posterior-to-anterior direction as shown in FIG. 3B. Thereafter, as shown in FIGS. 3C and 3D, the first piece 46 is maneuvered so that its upper surface is parallel to and in contact with the lower surface of the upper vertebra. An implantation tool can be used to hold the first piece 46 in place. The implantation tool can include one or more prongs that are received in the bores of the first piece 46 in order to hold the first piece 46 in place. It is preferred that at least part of the first piece 46 be urged laterally and be aligned so as to occupy space at the midsaggital region of the disk as shown in FIG. 3D. This can be achieved by moving the first piece 46 toward the center region of the disk. As will be apparent, this allows the multi-piece device, once assembled, to better support the weight that is placed upon it and to simulate the natural movement of the spine.

Using the same procedure, the second piece 48 of the multi-piece device is inserted through the opening as shown in FIG. 3E with the width of the second piece 48 being positioned along the diagonal of the remaining portion of the opening. Thereafter, the second piece 48 is maneuvered, using a tool similar to that used for the first piece 46, so that its lower surface is parallel to and in contact with the upper surface of the lower vertebra 16. Finally, a third piece 50 of the multi-piece device is inserted between the first and second pieces as shown in FIG. 3F. As will be further described herein, the third piece 50 includes an articular surface which allows the first and second pieces to move relative to each other.

With the inventive procedure, it is preferred that the pieces of a multi-piece device be inserted through the opening at the annulus in sequence according to size, i.e., width, with the piece with the largest width being inserted first. In this fashion, the multi-piece device can be readily assembled in situ, that is, within the disk region affected.

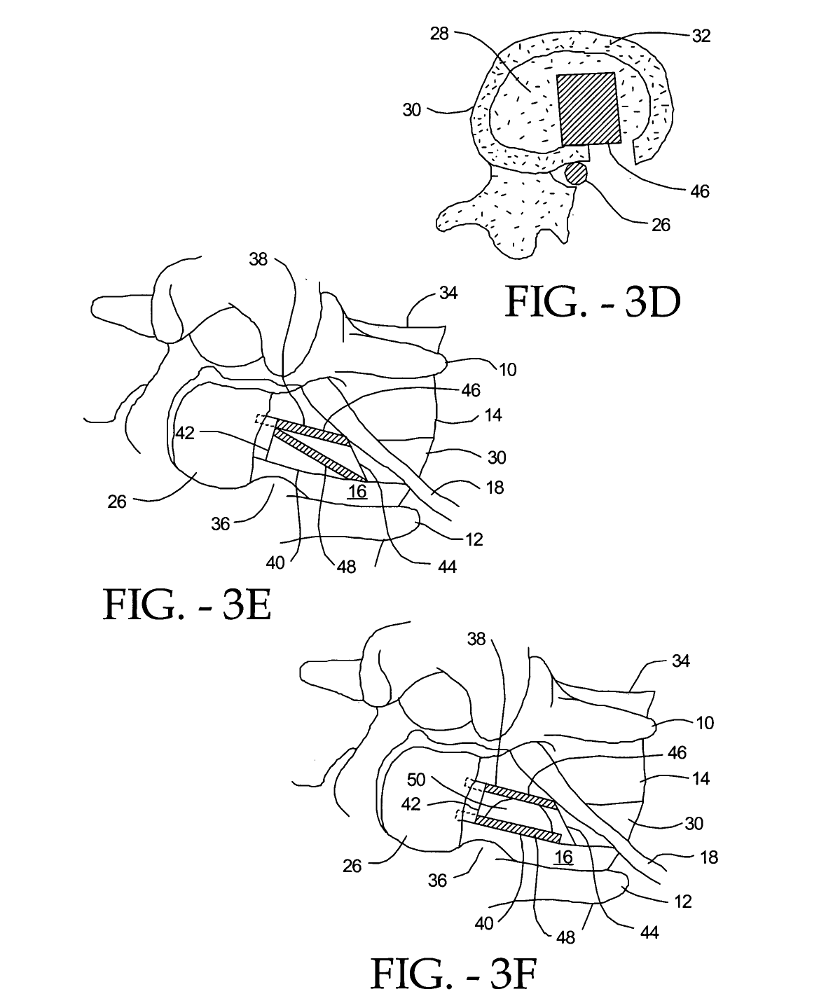

In cases where the device to be implanted does include a piece (or pieces) that has a particularly long width vis-à-vis the dimensions of the disk being treated or replaced, it may be necessary to remove bone from the vertebral body and/or process of the vertebra to accommodate the larger dimensions. As shown in FIG. 4, bone is removed, e.g., drilled, to create a slot 52 in the vertebral body 16. The combined length of the slot 52 and the diagonal of the opening is approximately equal to the width of the piece 54. As is apparent, the slot 52 and the diagonal are co-axial. The piece 54 is initially inserted through the slot 52 and the opening; thereafter, the piece 54 is urged laterally and aligned into the position as described previously (FIG. 3D).

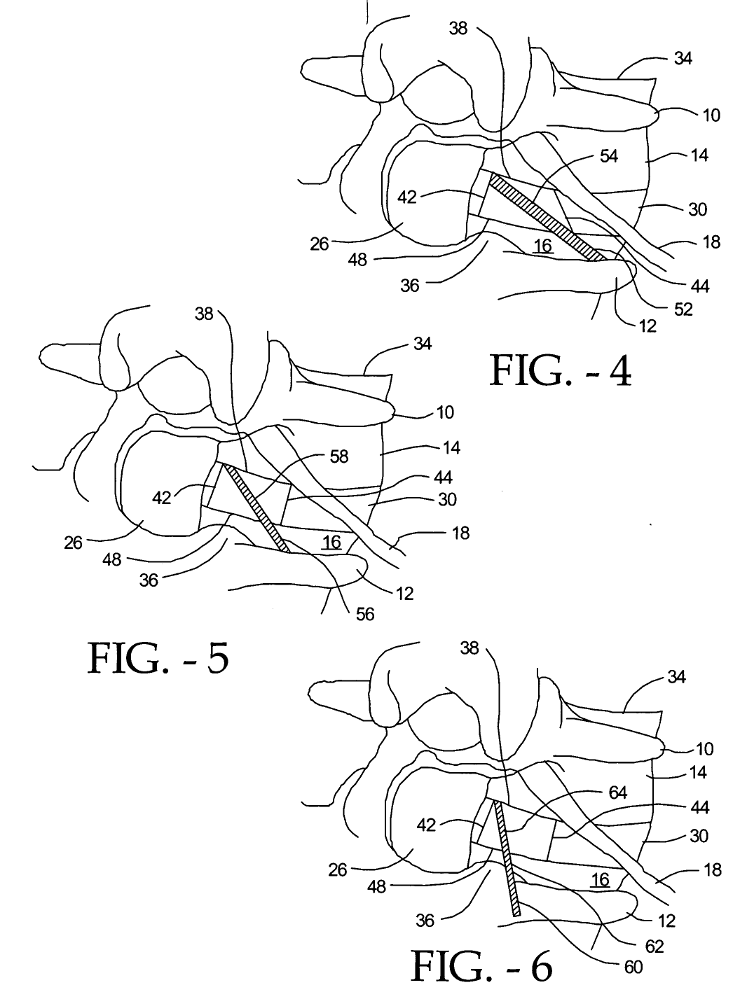

Similarly, as shown FIG. 5, bone is removed to create a slot 56 in the vertebral body 16. In this case, the slot 56 and the diagonal are not co-axial, rather, the slot 56 is drilled away from the corner of the rectangular opening. This procedure may be necessary in case of anatomical constraints. Piece 58 is initially inserted through the slot 56 and the opening. Thereafter, the piece 58 is urged laterally and aligned into the position as described previously.

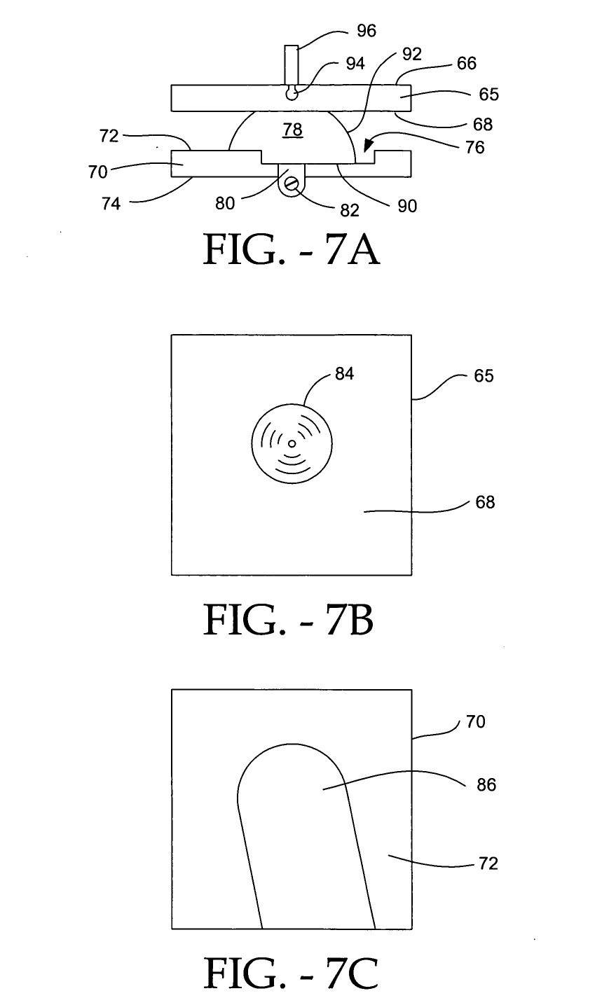

Finally, FIG. 6 shows an embodiment where a slot 60 is made in the pedicle 36 and a second slot 62 is made in the vertebral body 16. Piece 64 is initially inserted through the slots 60, 62 and the opening. Thereafter, the piece 64 is urged laterally and aligned into the position as described previously. The above slots 52, 56 and 62 are each also suitable for inserting a keel or similar apparatus into the vertebral body to support and anchor the piece 64 or any other part of the device as will be described below.

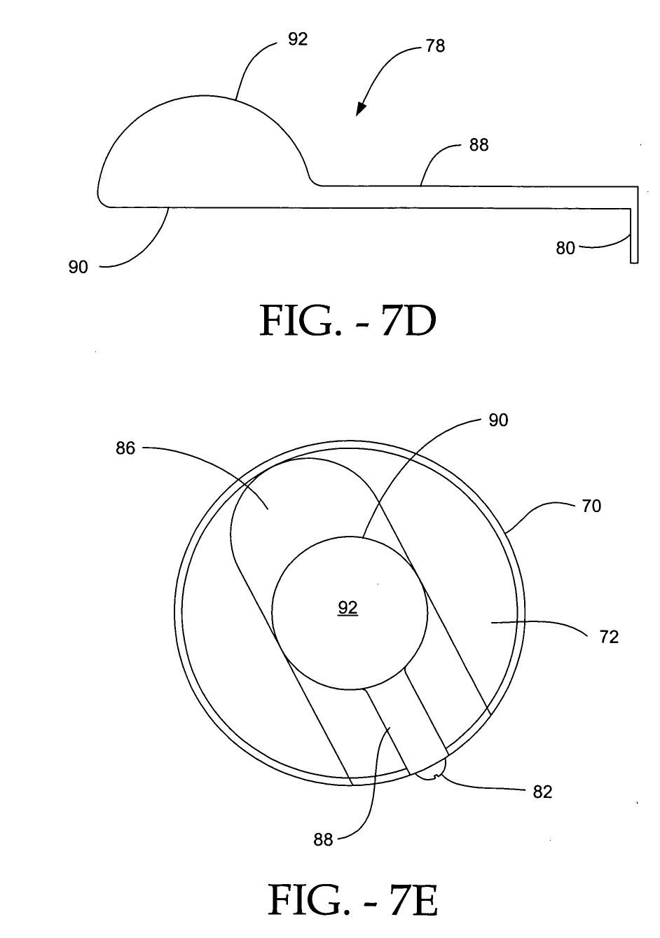

FIGS. 7A, 7B, 7C, and 7D illustrate a multi-piece device that can be assembled in situ with the above described posterior technique. (The device in FIG. 7A is shown in its neutral position where the first and second plates have not moved relative to each other.) The assembled implant includes a first plate 65 that is configured to mate with a first vertebra and a second plate 70 that is configured to mate with a second vertebra. The first or upper plate 65 has a first or upper surface 66 which abuts the vertebra body when the implant implanted. The first plate 65 can be secured to the upper vertebral body with a keel 96 that has a tongue 94 at its lower end. The keel 96 can have teeth on its upper surface. The tongue 94 fits snugly within a groove that is formed on the first surface 66. For a posterior approach, the teeth of the keel 96 would be pointed toward the posterior in order to aid in retaining the implant in place.

The second or lower surface 68 of the first plate 65 defines a recess 84 which has a concave surface with its center preferably at the middle of the first plate 64 as shown in FIG. 7B.

The second or lower plate 70 of the assembled multi-piece device has a first surface 74 which abuts the vertebra body when the implant is implanted. As shown in FIG. 7C, the second plate 70 also has a groove 86 that is formed on its second surface 72. The groove 86 has an entrance 76 on the posterior surface of the second plate 70 which defines a channel that traverses the approximate length of the second plate 70 from the posterior or surface to the anterior surface of the second plate 70. The axis along the center of the groove 86 is slanted so that while the entrance 76 is located towards the outer side of the posterior surface of the second plate 70, the groove moves toward the center of the second plate 70.

As shown in FIGS. 7A, 7D, and 7E, the assembled implant includes a third piece 78 that is positioned between the first and second plates 65, 70. The third piece has a lower circular base 90 that fits within the groove 86 of the second plate 70 and an upper articular surface 92 that has a convex exterior surface that substantially matches the contour of the exterior surface of the recess 84. The articular surface 92, which comes into slidable contact with the recess 84, allows the first plate 65 and second plate 70 to pivot and/or rotate relative to each other. The third piece 78 includes a neck 88 and strap 80 at the distal end. The length of the neck 88 is designed so that once the third piece 78 is properly positioned between the first and second plates 65, 70, the strap 80 contacts the posterior surface of second plate 70. The third piece 78 is secured to the lower vertebral body with a screw 82 which passes through an opening on the strap 80. To secured the second plate 70 to the lower vertebral body, a keel can also be employed as described above for securing the first plate 65.

As shown in FIG. 7A, the keel 96 is typically perpendicular to the upper surface 66 of upper plate 65. The keel thus projects into a cavity formed in the adjacent vertebral body 14. Preferably, the cavity defines an axis that is also perpendicular to the upper surface 66. In another embodiment, the keel 96 can be non-perpendicular to the upper surface 66 of upper plate 65 so that the corresponding cavity also has an axis that is not perpendicular. A similar arrangement of keel and cavity can also be employed to secured the lower plate 70 to vertebral body 16.

In another embodiment, the surface of keel 96 can be roughened in order that it can be securely received or anchored in the vertebra. In addition, the keel can have ports or holes formed therein so that bone can grow in the ports to further strengthen the attachment of the keel to the vertebra.

The complementary configurations of the recess 84 and the articular surface 92 allow the implant to simulate the natural motion of the spine. In a preferred embodiment, the articular surface 92 is a raised surface that is configured as a hemisphere and the corresponding recess 84 has a matching exterior contour shaped as a symmetrical circular cavity. The recess 84 covers only a portion of the surface area of the articular surface 92 at any given time. In this fashion, as the recess 84 traverses over different areas of the articular surface 92, the first plate 65, in turn, moves relative to the second plate 70. It is expected that the implant will restore natural movement to the patient thereby providing the patient with twisting or torsional movement as well as forward and backward bending motion, i.e., flexion and extension.

The level of movement can be tailored by appropriate design of the three pieces of the multi-piece implant, although it is understood the intervertebral implant functions in conjunction with the unaffected (or natural) structures of the spinal column. For example, the inter-plate distance between the first and second plates 65 and 70, that is, the distance between lower surface 68 of the first plate 65 and upper surface 72 of the second plate 70 determines the degree of forward and backward bending. The greater the inter-plate distance, the higher degree of movement possible, subject to other conditions. This inter-plate distance depends on the depth of the recess 84 and/or the height of the corresponding articular surface 92.

In assembling this multi-piece implant in situ, the spine is exposed and the first and second plates 65, 70 are then positioned between adjacent vertebrae by a posterior approach as described previously. Thereafter, the third piece 78 is inserted between the first and second plates 65, 70. Because the entrance 76 of the groove 86 is located on the outer lateral side of the second plate 70, the surgeon can readily maneuver the third piece 78 through the entrance 76 and into the groove 86.

The length of the neck 88 of the third piece 78 is selected so that when the third piece 78 is in positioned in the neutral position, the center of the recess 84 of the first piece 65 rests substantially on the center of the articular surface 92. Preferably, the recess 84 is fabricated to be in the middle of the first piece 65 however this position can be modified if desired. When the location of the recess 84 is changed, the groove 86 and the length of the neck 88 of the third piece 78 will be designed accordingly. It should be noted that, the third piece 78 can be positioned anywhere along the channel of groove 86 depending on the length of its neck 88. So, if the center of the modified recess 84 is still along the path of the channel of groove 86, the same second plate 70 and accompanying groove 86 can be employed and all that is needed is a third piece 78 with a neck 88 of the appropriate length.

It is to be understood that the embodiments of the invention can be made of titanium, stainless steel or other biocompatible materials, e.g., polymeric materials, that are suited for implantation in a patient. Metals are particularly suited given their physical and mechanical properties for carrying and spreading the physical load between the vertebrae.

Alternatively, the components of the implant can be made out of a polymer, and more specifically, the polymer is a thermoplastic. Still more specifically, the polymer is a polyketone known as polyetheretherketone (PEEK). Still more specifically, the material is PEEK 450G, which is an unfilled PEEK approved for medical implantation available from Victrex of Lancashire, Great Britain. Medical grade PEEK is available from Victrex Corporation under the product name PEEK-OPTIMA. Medical grade PEKK is available from Oxford Performance Materials under the name OXPEKK, and also from CoorsTek under the name BioPEKK. The components can be formed by extrusion, injection, compression molding and/or machining techniques. This material has appropriate physical and mechanical properties and is suitable for carrying and spreading the physical load between the spinous process. Further in this embodiment, the PEEK has the following additional approximate properties:

| Property | Value | |
|---|---|---|
| Density | 1.3 | g/cc |
| Rockwell M | 99 | |
| Rockwell R | 126 | |
| Tensile Strength | 97 | Mpa |
| Modulus of Elasticity | 3.5 | Gpa |
| Flexural Modulus | 4.1 | Gpa |

It should be noted that the material selected may also be filled. For example, other grades of PEEK are also available and contemplated, such as 30% glass-filled or 30% carbon-filled, provided such materials are cleared for use in implantable devices by the FDA, or other regulatory body. Glass-filled PEEK reduces the expansion rate and increases the flexural modulus of PEEK relative to that which is unfilled. The resulting product is known to be ideal for improved strength, stiffness, or stability. Carbon-filled PEEK is known to enhance the compressive strength and stiffness of PEEK and lower its expansion rate. Carbon-filled PEEK offers wear resistance and load carrying capability.

The components can also comprised of polyetherketoneketone (PEKK). Other material that can be used include polyetherketone (PEK), polyetherketoneether-ketoneketone (PEKEKK), and polyetheretherketoneketone (PEEKK), and, generally, a polyaryletheretherketone. Further, other polyketones can be used as well as other thermoplastics.

Reference to appropriate polymers that can be used in the components can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.”

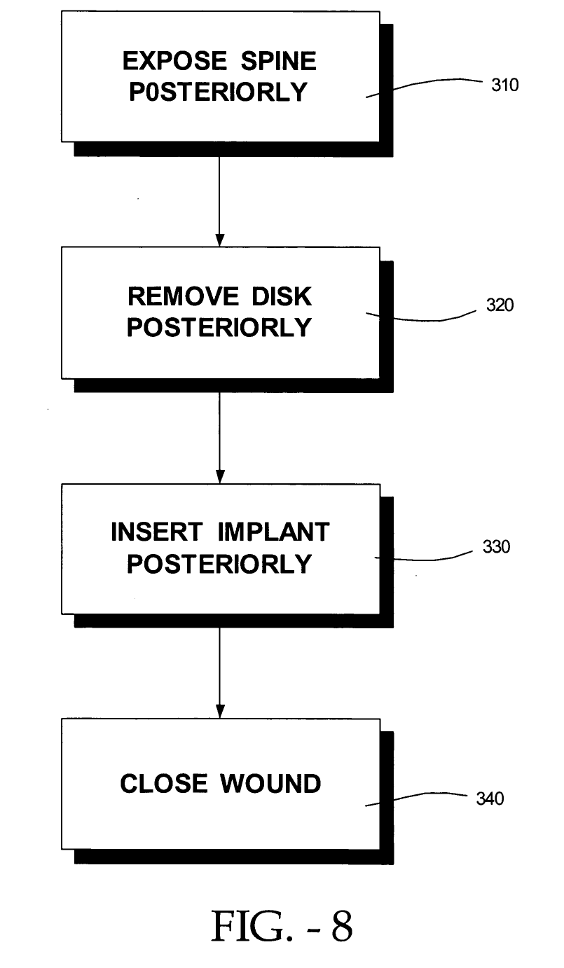

FIG. 8 is a block diagram showing the basic steps of the method of inserting the implant of the present invention. First the spine is exposed through a posterior access 310, then the intervertebral disk is removed 320 if necessary. The implant is then inserted posteriorly 330 between two vertebrae and the wound is closed 340.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and their practical application, thereby enabling others skilled in the art to understand the invention and the various embodiments and with various modifications that are suited to the particular use contemplated. It is intended that the scope of the invention be defined by the following claims and their equivalence.

What is claimed:

1. An implant for relieving pain associated with at least one of the spinal column and surrounding tissues and structures, which implant is positionable between a first vertebra and a second vertebra of the spinal column, wherein the first vertebra is located adjacent to and above the second vertebra, the implant comprising:
a first end plate having a first support surface and a top surface opposite the first support surface wherein the top surface includes means for securing the first end plate to the first vertebra;
a second end plate having a second support surface and a lower surface opposite the second support surface wherein the lower surface includes means for securing the second end plate to the second vertebra; and
a bearing member that is interposed between the first end plate and the second end plate wherein the bearing member has (i) a convex upper surface that is in contact with the first support surface and (ii) an opposite mounting surface that is in contact with the second support surface.

2. The implant of claim 1 wherein the bearing member has an articular surface that is in contact with the first support surface.

3. The implant of claim 1 wherein the means for securing the first end plate to the first vertebra comprises a first projection emanating from the top surface of the first end plate and wherein the first projection extends into a first cavity formed in the first vertebra.

4. The implant of claim 3 wherein the first cavity defines a first central axis that is not perpendicular to the plane defined by the first support surface.

5. The implant of claim 3 wherein the means for securing the second end plate to the second vertebra comprises a second projection emanating from the lower surface of the second end plate and wherein the second projection extends into a second cavity formed in the second vertebra.

6. The implant of claim 5 wherein the second cavity defines a second central axis that is not perpendicular to the plane defined by the second support surface.

7. The implant of claim 1 wherein the means for securing the first end plate comprises a first keel extending from the top surface, the first keel adapted to penetrate into the first vertebra and wherein the means for securing the second end plate comprises a second keel extending from the lower surface, the second keel adapted to penetrate into the second vertebra.

8. The implant of claim 7 wherein the first keel extends at an angle from the top surface and the second keel extends at an angle from the lower surface.

9. The implant of claim 7 wherein the first keel extends substantially perpendicular from the top surface and the second keel extends substantially perpendicular from the lower surface.

10. The implant of claim 7 wherein the first and second keels are each sharpened in order to penetrate a vertebra.

11. The implant of claim 7 wherein the first and second keels are each roughened in order to be securely received in a vertebra.

12. The implant of claim 7 wherein the first and second keels each has at least one port which is adapted to receive bone which grows there through.

13. The implant of claim 7 wherein the first and second keels are each includes means for preventing the keel from backing out once the keel is inserted in a vertebra.

14. The implant of claim 1 wherein the first end plate, second end plate, and bearing member are each made of metal.

15. The implant of claim 14 wherein the first end plate, second end plate, and bearing member are each made of a polymer.

16. The implant of claim 1 wherein the second support surface of the second end plate defines a channel into which the bearing member is inserted.

17. The implant of claim 16 wherein the channel extends from one side of the implant to the opposite side.

18. The implant of claim 17 wherein the length of the channel is sufficient to accommodate the bearing member at multiple positions along the length of the channel.

19. The implant of claim 16 wherein the channel defines an ingrowth surface.

20. The implant of claim 16 wherein the bearing member has an articular surface that is in contact with the first support surface.

21. The implant of claim 20 wherein the bearing member has a spherical base and a convex upper surface that is in contact with the first support surface.

22. The implant of claim 16 further comprising means for selectively securing the bearing member to the channel in one of a plurality of positions in order to adjust the position on the first support surface that is in contact with articular surface of the bearing member.

23. The implant of claim 1 further comprising means for securing the bearing member between the first end plate and the second end plate.

24. The implant of claim 23 wherein the bearing member includes a lip portion that extends from a side of the bearing member and wherein the lip portion defines a hole adapted to receive a screw or pin that anchors the lip portion to a side of the first or second vertebra.

25. The implant of claim 16 further comprising means for selectively securing the bearing member to channel in one of a plurality of positions.

26. An implant for relieving pain associated with at least one of the spinal column and surrounding tissues and structures, which implant is positionable between a first vertebra and a second vertebra of the spinal column, wherein the first vertebra is located adjacent to and above the second vertebra, the implant comprising:
a first end plate having a first support surface and a top surface opposite the first support surface wherein the top surface;
a second end plate having a second support surface and a lower surface opposite the second support surface; and
a bearing member that is interposed between the first end plate and the second end plate wherein the bearing member has (i) a convex upper surface that is in contact with the first support surface and (ii) an opposite mounting surface that is in contact with the second support surface and wherein the second support surface defines a channel into which the bearing is inserted.

27. A method of implanting a multiple piece device in situ between an upper and a lower vertebral body in a spine, the method comprising:
(a) accessing an affected region of the spine posteriorly by performing a minor posterior annulotomy;
(b) removing at least a portion of the affected region to create an exposed area in the region which is defined by a region length and a region width;
(c) positioning a first piece of the device into the affected region, wherein the first piece comprises a first plate having a first plate length and a first plate width, with the first plate width being longer than the region length, and wherein the first plate is positioned so that its upper surface contacts the upper vertebral body;
(d) positioning a second piece of the device into the affected region, wherein the second piece comprises a second plate having a second plate length and a second plate width, with the second plate width being longer than the region length, and wherein the second plate is positioned so that its lower surface contacts the lower vertebral body; and
(e) inserting a third piece of the device between the first and second pieces wherein the third piece comprises an articulating surface that supports the first plate and a lower surface that rests on the second plate.

28. The method of claim 27 wherein the exposed area is defined by a diagonal that is equal to or equal than either of the widths of the first and second plates and wherein:
step (c) comprises the steps of (i) placing the first plate along a diagonal of the region and (ii) aligning the first plate so that its upper surface is in contact with the upper vertebral body; and
step (d) comprises the steps of (i) placing the second plate along a diagonal of the region and (ii) aligning the second plate so that its lower surface is in contact with the lower vertebral body.

29. The method of claim 27 wherein the articulating surface has a convex contour and the first plate has a groove and at least a portion of the convex contour is in slidable contact with a groove surface.

30. The method of claim 27 wherein the exposed area is defined by a diagonal that is shorter than at least one of the widths of the first and second plates and wherein the method comprises the steps of (i) removing a first channel from either the upper or lower vertebral body such that the first channel has an aperture on a surface of either the upper or lower vertebral body, (ii) maneuvering the first plate into the region, and (iii) maneuvering the second plate into the region.

31. The method of claim 27 wherein the exposed area is defined by a diagonal that is shorter than the widths of the first and second plates and wherein the method comprises the step of forming a first channel within either the upper or lower vertebral body such that the first channel has an aperture on a surface of either the upper or lower vertebral body and wherein:
step (c) comprises inserting the first plate into the region and into the first channel and maneuvering the first plate into region, and
step (d) comprises inserting the second plate into the region and into the first channel and maneuvering the second plate into region.

32. The method of claim 31 wherein the first channel defines a slot with an axis that is not perpendicular to the surface of either the upper or lower vertebral body in which the first channel is formed.

33. The method of claim 31 wherein step (c) further comprises securing the first plate to the upper vertebral body and step (d) further comprises securing the second plate to the lower vertebral body.

34. The method of claim 32 wherein step (c) further comprises securing the first plate to the upper vertebral body and step (d) further comprises securing the second plate to the lower vertebral body.

35. The method of claim 31 wherein the exposed area is defined by a diagonal that is shorter than the widths of the first and second plates, and wherein:
step (c) comprises the steps of (i) forming a first channel within the upper vertebral body such that the first channel has a first aperture on a first surface of the upper vertebral body, (ii) inserting the first plate into the region and into the first channel, and (iii) maneuvering the first plate into region, and
step (d) comprises the steps of (i) forming a second channel within the lower vertebral body such that the second channel has a second aperture on a second surface of the lower vertebral body, (ii) inserting the second plate into the region and into the second channel, and (iii) maneuvering the second plate into region.

36. The method of claim 35 wherein the first channel defines a slot with an axis that is not perpendicular to the surface of either the upper or lower vertebral body in which the first channel is formed.

37. The method of claim 35 wherein step (c) further comprises securing the first plate to the upper vertebral body and step (d) further comprises securing the second plate to the lower vertebral body.

38. The method of claim 36 wherein step (c) further comprises securing the first plate to the upper vertebral body and step (d) further comprises securing the second plate to the lower vertebral body.

39. A method of relieving pain associated with at least one of the spinal column and associated tissues and structures comprising the steps of:
(a) posteriority exposing an annulus comprised of fibrous tissue positioned between first and second adjacent vertebrae;
(b) removing at least a portion of the fibrous tissue from the annulus; and
(c) positioning an implant into the annulus which comprises the steps of:
(i) inserting a first end plate having a first support surface and a top surface opposite the first support surface wherein the top surface;
(ii) inserting a second end plate having a second support surface and a lower surface opposite the second support surface wherein the lower surface wherein the second support surface includes a channel; and
(iii) positioning a bearing member between the first end plate and the second end plate wherein the bearing member has (i) a convex upper surface that is in contact with the first support surface and (ii) an opposite mounting surface that is in contact with the second support surface wherein the bearing member is inserted through the channel of the second support surface.
